(19) United States
(12) Patent Application Publication
(10) Pub. No.: US 2020/0139155 A1
(43) Pub. Date: May 7, 2020

(54) CATHETER APPARATUS AND BRACHYTHERAPY SYSTEM

(71) Applicant: BRAXX BIOTECH CO., LTD, Taipei (TW)

(72) Inventors: Kai-Lin Yang, Taipei (TW); Hsuan-Mien Wang, Taipei (TW); Wei-Jer Chang, New Taipei (TW); Jeng-Yu Chou, Yilan County (TW); Tsung-Yu Lai, Changhua County (TW); Ming-Cheng Chen, New Taipei (TW)

(21) Appl. No.: 16/629,317

(22) PCT Filed: Dec. 15, 2017
(86) PCT No.: PCT/CN2017/116407

§ 371 Date: Jan. 8, 2020

(51) Int. Cl. A61N 5/10; A61M 25/00; A61M 25/10; A61B 6/03; A61B 6/00; A61B 5/055

(52) U.S. Cl. CPC A61N 5/1007; A61N 2005/1008; A61M 25/0068; A61M 25/1011; A61M 25/1002; A61M 25/10184; A61B 6/032; A61B 6/037; A61B 6/487; A61B 5/055; A61M 2025/0034; A61M 2025/1015; A61M 2025/1004; A61M 2025/1061; A61M 2025/1086; A61M 2205/02; A61M 25/0026

(57) ABSTRACT

The invention provides a catheter apparatus, which includes an integrally formed multi-lumen pipeline structure having a proximal end direction and a distal end direction, wherein the multi-lumen pipeline structure includes a tubular structure and multiple fluid flow pipe structures, and the tubular structure and the multiple fluid flow pipe structures are disposed along a first axial direction of the multi-lumen pipeline structure; at least one pipe sleeve membrane element wrapped on the outer periphery of the multi-lumen pipeline structure, wherein the at least one pipe sleeve membrane element includes a strengthening structure and/or a buffering structure; and a pointed end which is jointed with the multi-lumen pipeline structure and is tightly fastened with the multi-lumen pipeline structure. The invention further provides a brachytherapy system using the catheter apparatus, which can be used for treating intracavitary tumors such as esophageal cancer or the like.

### TECHNICAL FIELD

The present application relates to a catheter apparatus for use in brachytherapy, particularly a catheter apparatus and a brachytherapy system with strengthening and buffering structures for esophageal cancer.

### BACKGROUND

Brachytherapy is a radiation therapy for intracavitary tumors. Brachytherapy for a tumor is by inserting a catheter into a body cavity or an organ, placing the catheter close to the surroundings of the tumor tissue, then passing a radioactive source into the catheter with an afterload-type therapeutic instrument, and keeping the radioactive source in the tumor area. The high-energy radiation, transferred in the form of light wave or high-speed particle, will destroy the tumor cells and inhibit the tumor cells growth.

Esophageal cancer is a malignant tumor grown in esophagus. Side effects apparently occur with the accumulated radiation dosages during treatment of esophageal cancer with brachytherapy. These side effects of radiation therapy, such as radiation pneumonitis, radiation esophagitis, or acute bleeding in the esophagus, are related to the area exposed to the radiation and radiation dose. The radiation intensity is inversely proportional to the square of the distance from the radioactive source, the closer to the radioactive source the normal tissues are, the higher dose they receive, and the greater the side effect is. As shown in FIG. 1 (Hitoshi Ikushima, Radiation therapy: state of the art and the future, The Journal of Medical Investigation Vol. 57 February 2010). Besides, brachytherapy is a course of therapy that requires consistency and reproducibility over several treatments and requires precise positioning to ensure that the tumor receives a consistent therapeutic dose in each treatment. Due to internal movement of human organs (for example, the thoracic cavity is expanded or contracted with the movement of diaphragm when breathing and all organs located in the thoracic cavity are moved) the normal tissue will be exposed to higher radiation dose resulting in inaccurate radiotherapy when the relative position of the radioactive source and the tumor isn't fixed precisely. As shown in FIG. 2 (Hitoshi Ikushima, Radiation therapy: state of the art and the future, The Journal of Medical Investigation Vol. 57 February 2010).

Because of the difference in each patient's body cavity/organ and tumor size, the biggest challenge in clinical personalized medicine is providing the optimal therapeutic dose according to the situation of normal tissues, tumor of each patient and the radioactive source. Nasogastric tube is usually used for current clinical treatment. However, its diameter is relatively small, its fixation effect is poor, and it is unable to keep the radioactive source in the center of esophagus. Since high radiation dose is required for esophageal brachytherapy, when the nasogastric tube is attached to the esophageal wall randomly, it causes the radioactive source to be too close to the normal esophageal wall and thereby resulting in excessive dose and radiation hotspots, which cause serious side effects. This affects the doctors' willingness to use nasogastric tube for esophageal brachytherapy. The existing catheter used for brachytherapy of esophageal cancer is inserted into the esophagus from the mouth and causes discomfort to the patient.

For instance, the Bonvoisin-Gerard Esophageal Applicator product by Elekta dilates the body cavity by thickening the whole section of the catheter. The radioactive source is placed in the middle of the thickened catheter. But since the dose of the radioactive source is inversely proportional to the square of the distance, more normal tissue areas will be irradiated and resulted in side effects when the tumor growth is in the superficial area. Meanwhile, the dilation of the whole esophagus without difference will affect the dose treatment planning during radiotherapy and can't provide the optimal dose conformity. At the place where the tumor is relatively large and the esophagus is narrow, the thickened catheter may rub against the tumor and cause bleeding. Furthermore, since the use of catheter with the entire section thickened and without undulation will easily result in slippery of the catheter with poor fixation in the smooth and peristaltic esophagus.

Taking the catheter disclosed in Chinese Patent Publication No. 202387089U as an example, the catheter has a catheter body, an imaging ring, at least two balloons, a balloon lumen, a balloon filling channel, a balloon injection port, a guide wire (guide line) channel, and a guide wire channel port. The balloons are of the same diameter and in a long cylindrical shape. During treatment, the end balloon is inflated first, and then the adjacent balloon is inflated in turns; thereby on the basis of the end balloon expansion, the entire length of the balloon can be directly extended without changing the balloon catheter, so as to achieve fixation of the tumor which length is above 3 cm. However, the balloons are additional long cylindrical balloons. If the inflation is insufficient for standard supporting, the balloon may not be uniformly expanded and the radioactive source cannot be maintained in the center of the catheter. As a result, it will reduce the reproducibility of the treatment planning. Besides, the operational procedure will be increased due to the necessity of the assistance of the guide wire.

To avoid the occurrence of serious side effects, American Brachytherapy Society suggests the diameter of the applicator for brachytherapy should be at least 10 mm. Elekta and Varian companies have also developed such thickened therapeutic applicators. In clinical, the applicator is inserted into the body cavity guided by a guide wire and an endoscope, and requires operation by a gastroenterologist. The application of the guide wire increases the operational procedure. Furthermore, placing the applicator from the mouth will easily induce vomiting reflex or swallowing reaction, which changes the catheter position and causes discomfort to the patient. Therefore, sedative or anesthesia is needed and the patients must also lie on their side. When obtaining the tumor imaging data, the doctor will decide the treatment plan of the patient (for determining the position and duration of the radioactive source), and then move the patient to the bed for brachytherapy. At this time, as long as the bent curvature of the patient changes, the applicability of the doctor's treatment planning is reduced, resulting in the treatment being inaccurate, adding operational inconvenience and risk.

Taking another applicator disclosed in U.S. Patent Publication No. 20170173362A as an example, the catheter has a proximal balloon, a distal balloon and an independently inflatable middle balloon which is placed between the proximal and the distal balloons. The catheter will prevent the healthy tissue area which is located besides the patient's tumor from radiation dose and reduce the side effects by independently inflatable balloons. However, the applicator doesn't solve the issue of wound bleeding caused by radiation hotspots due to uneven expansion of the balloons and deviation of radioactive source from the center of the esophagus. Besides, it is necessary to pass the guide wire through the guidewire lumen and the opening end of the applicator during insertion of the applicator into patient's esophagus. The problem of increasing operational procedure with guide wire is not solved. That doesn't increase the medical personnel's willingness for using it.

For saving the gastroenterologist's operational time, a similarly flexible catheter is disclosed in U.S. Patent Publication No. US20100185173A1. The catheter with medical balloons has two inflatable balloons and a removable inner tube. The catheter with deflated balloons can be inserted into the patient's esophagus from the nasal throat by a non-specialist. After the inflatable balloon is positioned at the area for treatment, then the radioactive source is introduced. However, the catheter doesn't solve the problems of wound bleeding caused by radiation hotspots due to insufficient inflation of the balloons and deviation of the radioactive source from the center of the esophagus. Besides, when the balloon expands to a certain volume with insufficient supporting force to support the esophageal wall, optimal treatment dose can't be provided according to the situation of each patient's normal tissue, tumor and radioactive source.

The catheter disclosed in Chinese Patent Publication No. CN2345224Y is used for esophageal treatment. The catheter has one suction chamber, one drug liquid drop-in chamber, two balloon inflation chamber, two balloons and one closed solid blunt conical head. The catheter's two inflated balloons are blocking at two ends of the tumor after inserting into the esophagus from a nasal cavity. Saliva is removed from the suction chamber and chemotherapy or immunotherapy drug is administered from the drug liquid drop-in chamber. The catheter has a length of 100-150 mm and provides enough space for keeping the drug liquid so as to reduce side effects. However, the catheter requires an extra fixing stand on the nose for preventing slippery of the catheter in the esophagus. It means that the catheter has insufficient supporting force to support the esophageal wall. Hence, it can't provide the optimal radiation dose to treat the tumor, normal tissue and radioactive source during brachytherapy. It may also be dangerous of causing bleeding due to the rubbing of the balloons with the tumor when treating diffuse tumors. Besides, it may damage the body when the conical head falls off in the esophagus during the process of surgery.

The existing catheters have the above disadvantages. Therefore, designing a catheter which can provide high treatment doses for killing tumor cells, reducing relapse ratio, protecting the normal tissue, avoiding radiation hotspots during brachytherapy, reducing side effects, without changing the physician's habit, without the aid of guide wire, avoiding multiple operations when there are multiple tumors or diffuse tumors, avoiding displacement of the catheter in esophagus caused by the patient's movement, saving the physician's energy and reducing patient's discomfort, are the actual problems to be solved.

### SUMMARY

The present application provides a catheter apparatus, including an integrally formed multi-lumen pipeline structure, having a proximal end direction and a distal end direction, wherein the multi-lumen pipeline structure includes a tubular structure and multiple fluid flow pipe structures, the tubular structure and multiple fluid flow pipe structures are disposed along a first axial direction of the multi-lumen pipeline structure; and at least one pipe sleeve membrane element wrapping around an outer periphery of the multi-lumen pipeline structure, wherein the at least one pipe sleeve membrane element includes a strengthening structure and/or a buffering structure; and a pointed end, which is jointed to the multi-lumen pipeline structure and is firmly fixed with the multi-lumen pipeline structure.

Based on the above concept, wherein the catheter apparatus is provided with multiple outer ring elements disposed on the outer periphery of the pipe sleeve membrane element, the multiple outer ring elements are used for fastening the pipe sleeve membrane element with the multi-lumen pipeline structure to form multiple sleeve membrane structures.

Based on the above concept, wherein the sleeve membrane structure is a cylindrical or a waist drum shaped structure surrounding the multi-lumen pipeline structure.

Based on the above concept, wherein the sleeve membrane structure has a membrane thickness, a middle segment and two side segments, the membrane thickness decreases progressively from the middle segment to the two side segments.

Based on the above concept, wherein a quantity of the pipe sleeve membrane elements is more than 1; a quantity of the fluid flow pipe structure is more than 3; a quantity of the sleeve membrane structure is more than 3.

Based on the above concept, wherein the pointed end is integrally formed with the multi-lumen pipeline structure.

Based on the above concept, wherein the pointed end is a cone or truncated cone structure jointed with the multi-lumen pipeline structure.

Based on the above concept, wherein the pointed end is a closed structure, and the pointed end includes a material which can absorb radiation.

Based on the above concept, wherein the pointed end includes a main joint structure which is used for fixing to a sub-joint structure of the multi-lumen pipeline structure.

Based on the above concept, wherein the main joint structure and the sub-joint structure are corresponding to each other in the form of a latch, a buckle or a screw.

Based on the above concept, wherein the strengthening structure is disposed on an inner or outer side of the pipe sleeve membrane element, making each of the sleeve membrane structures to be inflated uniformly at a constant speed from an axis to a surrounding radially.

Based on the above concept, wherein the strengthening structure is at least one strip-shaped structure or multiple dot-shaped structures distributed on the sleeve membrane structure.

Based on the above concept, wherein the strip-shaped structure is a symmetrical, a parallel, a criss-cross and/or a non-continuous structure.

Based on the above concept, wherein the buffering structure is a recessed, a protruding or a folding structure disposed on the outer periphery of the pipe sleeve membrane element, making the sleeve membrane structure release pressure uniformly during the beginning of inflation.

Based on the above concept, wherein the multiple fluid flow pipe structures are provided with a control element in the proximal end direction, and the control element is configured for individually and independently inflating and deflating the sleeve membrane structure connected to the fluid flow pipe structure in the distal end direction.

Based on the above concept, wherein the multiple fluid flow pipe structures are provided with multiple control elements in the proximal end direction, and the multiple control elements are disposed individually and independently on the multiple fluid flow pipe structures in the proximal direction; each control element is configured for independently inflating and deflating the sleeve membrane structure connected to the fluid flow pipe structure in the distal end direction.

Based on the above concept, wherein the multiple fluid flow pipe structures each are provided with an independent connecting structure which are connected with different positions of the sleeve membrane structures respectively, transferring fluid from the fluid flow pipe structures to different sleeve membrane structures through respective independent connecting structure.

Based on the above concept, wherein the independent connecting structure is a channel or an opening structure.

The present application also provides a brachyherapy system, including an afterload-type therapeutic instrument; the catheter apparatus connected to the afterload-type therapeutic instrument; and a radiation therapy source, released from the afterload-type therapeutic instrument to the tubular structure of the catheter apparatus.

Based on the above concept, further provided with a tumor imaging instrument, wherein the afterload-type therapeutic instrument releases the radiation therapy source to the positions of the sleeve membrane structures of the tubular structure according to determination by the tumor imaging instrument.

Based on the above concept, wherein the tumor imaging instrument includes one or more of X-ray imaging, fluoroscope, computed tomography scan, positron tomography scan, single photon emission tomography imaging, and nuclear magnetic resonance imaging.

Based on the above concept, wherein the brachytherapy system is used for treatment of esophageal cancer or other intracavitary tumors.

### BRIEF DESCRIPTION OF DRAWINGS

FIG. 1 shows the relationship between radiotherapy dose and tissue toxicity.

FIG. 2 is a schematic view of the radiation area, and deviation and displacement of external radiotherapy.

FIG. 3 is an illustrative structural diagram of the catheter apparatus according to one embodiment of the present application.

FIG. 4 is an illustrative diagram showing the cross-section A-A of the catheter apparatus according to one embodiment of the present application.

FIG. 5 is an illustrative structural diagram of the catheter apparatus according to one embodiment of the present application.

FIG. 6(a) is an illustrative structural diagram of the sleeve membrane structure according to one embodiment of the present application.

FIG. 6(b) is an illustrative structural diagram of the sleeve membrane structure according to one embodiment of the present application.

FIG. 6(c) is an illustrative side view of the sleeve membrane structure according to one embodiment of the present application.

FIG. 7 is an illustrative structural diagram of the strengthening structure according to one embodiment of the present application.

FIG. 8 is an illustrative structural diagram of the strengthening structure according to one embodiment of the present application.

FIG. 9(a) is an illustrative structural diagram of the strengthening structure according to one embodiment of the present application.

FIG. 9(b) is an illustrative structural diagram of the strengthening structure according to one embodiment of the present application.

FIG. 9(c) is an illustrative structural diagram of the strengthening structure according to one embodiment of the present application.

FIG. 10(a) is an illustrative structural diagram of the strengthening structure according to one embodiment of the present application.

FIG. 10(b) is an illustrative structural diagram of the strengthening structure according to one embodiment of the present application.

FIG. 11(a) is an illustrative structural diagram of the strengthening structure according to one embodiment of the present application.

FIG. 11(b) is an illustrative structural diagram of the strengthening structure according to one embodiment of the present application.

FIG. 12(a) is an illustrative structural diagram of the strengthening structure according to one embodiment of the present application.

FIG. 12(b) is an illustrative structural diagram of the strengthening structure according to one embodiment of the present application.

FIG. 13(a) is an illustrative structural diagram of the strengthening structure and the buffering structure according to one embodiment of the present application.

FIG. 13(b) is an illustrative structural diagram of the strengthening structure and the buffering structure according to one embodiment of the present application.

FIG. 13(c) is an illustrative diagram showing the cross-section B-B of the strengthening structure according to one embodiment of the present application.

FIG. 14(a) is an illustrative structural diagram of the strengthening structure and the buffering structure according to one embodiment of the present application.

FIG. 14(b) is an illustrative structural diagram of the strengthening structure and the buffering structure according to one embodiment of the present application.

FIG. 14(c) is an illustrative diagram showing the cross-section C-C of the strengthening structure according to one embodiment of the present application.

FIG. 15(a) is an illustrative structural diagram of the strengthening structure and the buffering structure according to one embodiment of the present application.

FIG. 15(b) is an illustrative structural diagram of the strengthening structure and the buffering structure according to one embodiment of the present application.

FIG. 15(c) is an illustrative diagram showing the cross-section D-D of the strengthening structure according to one embodiment of the present application.

FIG. 16(a) is an illustrative side view showing the buffering structure before inflation according to one embodiment of the present application.

FIG. 16(b) is an illustrative side view showing the buffering structure after inflation according to one embodiment of the present application.

FIG. 16(c) is an illustrative perspective view showing the buffering structure after inflation according to one embodiment of the present application.

FIG. 17(a) is an illustrative side view showing the buffering structure before inflation according to one embodiment of the present application.

FIG. 17(b) is an illustrative side view showing the buffering structure after inflation according to one embodiment of the present application.

FIG. 17(c) is an illustrative perspective view showing the buffering structure after inflation according to one embodiment of the present application.

FIG. 18 is an illustrative diagram showing each sleeve membrane structure inflated independently according to the present application.

FIG. 19 is an illustrative diagram showing the sleeve membrane structures of the catheter apparatus of the present application inflated and deflated independently to conform in shape with the tumor.

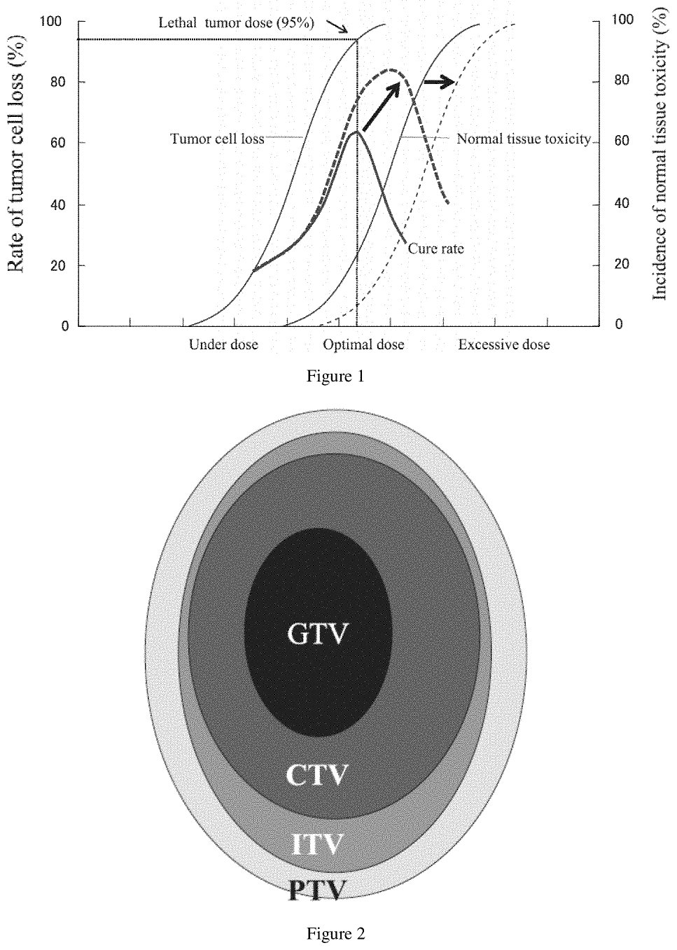

### DETAILED DESCRIPTION

Unless otherwise defined, all technical and scientific terms in the context represent the same meanings which a person having ordinary skill in the art comprehends with.

The “catheter apparatus” of the present application can be explained according to the following description of the embodiments, which allows one skilled in the art to understand the spirit of creation and make the catheter apparatus.

The modes of implementation of the present application are not limited by the embodiments.

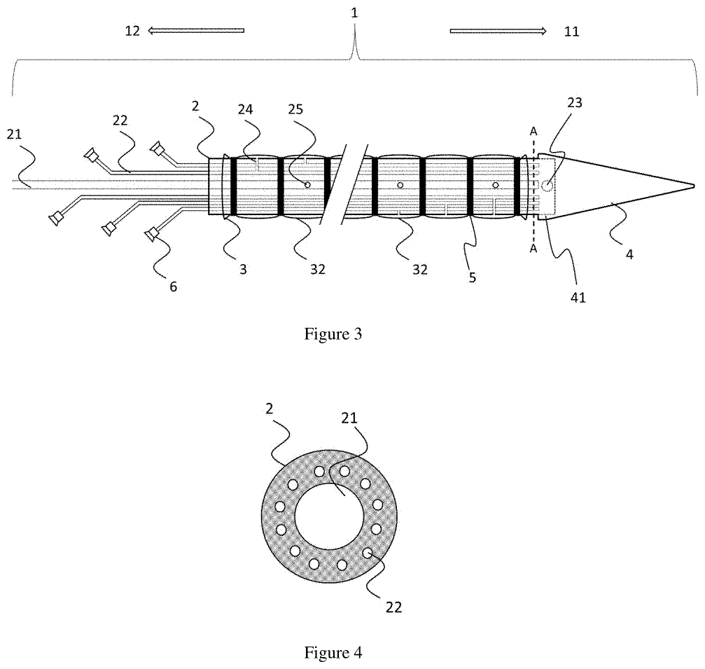

FIG. 3 shows an illustrative diagram of the catheter apparatus 1 according to one embodiment of the present application, and FIG. 4 shows an illustrative diagram of the cross-section A-A of the multi-lumen pipeline structure of the catheter apparatus according to one embodiment of the present application

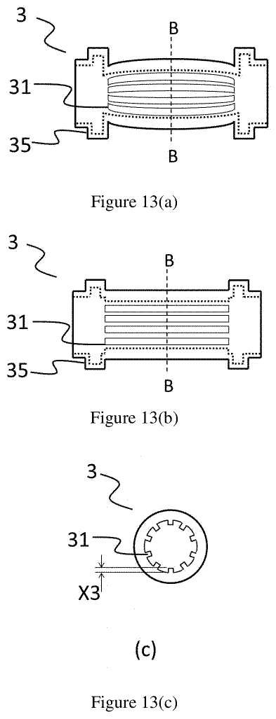

The catheter apparatus 1 includes an integrally formed multi-lumen pipeline structure 2, and the multi-lumen pipeline structure 2 includes a tubular structure 21 and multiple fluid flow pipe structures 22. The tubular structure 21 disposed along a first axial direction of the multi-lumen pipeline structure 2 is in the middle of the catheter apparatus 1 for placing a radioactive source 25. The multiple flow pipe structures 22 disposed along the first axial direction of the multi-lumen pipeline structure 2 are distributed around the tubular structure 21 (the “first axial direction” in the embodiment of the present application is the direction by taking the long side of the catheter apparatus as the axis). The multi-lumen pipeline structure 2 is wrapped by at least one pipe sleeve membrane element 3 on the outer periphery. Multiple outer ring elements 5 are disposed on the outer periphery of the pipe sleeve membrane element 3. The multiple outer ring elements 5 are used for fastening the pipe sleeve membrane element 3 with the multi-lumen pipeline structure 2 and form multiple sleeve membrane structures 32. The inner or outer side of the pipe sleeve membrane element 3 are provided with a strengthening structure 31 and/or a buffering structure 35 (shown in FIG. 13).

The multiple fluid flow pipe structures 22 in the multi-lumen pipeline structure 2 are of the same length. An independent connecting structure 24 is provided on each of the multiple fluid flow pipe structures 22 at its distal end direction 11 and an independent control element 6 at its proximal end direction 12. Different control elements 6 may transfer the fluid (not shown) into the fluid flow pipe structure 22 respectively. The fluid may flow to the end of each fluid flow pipe structure 22 at the distal end 11, or it may flow to the different independent connecting structure 24 disposed in the middle of the fluid flow pipe structure 22. It will fill in the inner space of different sleeve membrane structure 32 and inflate or deflate the sleeve membrane structure 32 for the effect of positioning. Since different independent connecting structures 24 are disposed at different positions of the sleeve membrane structure 32 respectively, the fluid is transferred to the different sleeve membrane structures 32 from different fluid flow pipe structures 22 through respective independent connecting structures 24 and makes the sleeve membrane structures 32 to inflate and deflate and achieve individual adjustment of the degree of inflation and deflation. A pointed end 4 jointed to the multi-lumen pipeline structure 2 is disposed in the multi-lumen pipeline structure 2 in the distal end direction 11.

In some embodiments, the pointed end 4 has a main joint structure 41. The multi-lumen pipeline structure 2 has a sub-joint structure 23 in the distal end direction 11. The pointed end 4 is jointed with the multi-lumen pipeline structure 2 stably by the main joint structure 41 and the sub-joint structure 23. That makes the pointed end 4 and multi-lumen pipeline structure 2 fixed tightly without falling off easily. In some embodiments, the main joint structure 41 of the pointed end 4 and the sub-joint structure 23 of the multi-lumen pipeline structure 2 can be corresponding to each other in the form of a latch, a buckle or a screw, that may resist from pushing force, pulling force or other external force from every direction and can avoid the pointed end falling off during treatment. In some embodiments, the pointed end 4 includes a material which can absorb radiation for confirming where the catheter apparatus 1 is located in the human body.

In some embodiments, the control element 6 can be a medical pump, a syringe, or an injection device. In other embodiments, the control element 6 can be a check valve or a two-way valve.

In some embodiments, the multiple fluid flow pipe structure 22 can be connected with a single control element (not shown), such as an air pump controlled by a computer, and control the sleeve membrane structures 32 in distal end direction 11 by valves independently.

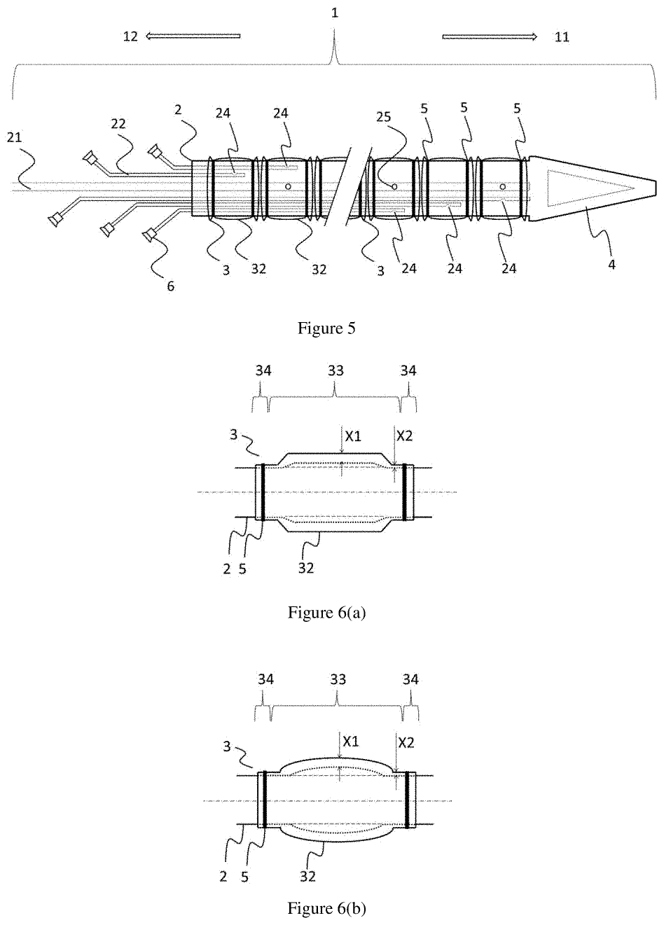

FIG. 5 is an illustrative structural diagram of the catheter apparatus 1 according to another embodiment of the present application.

In some embodiments, the integrally formed multi-lumen pipeline structure 2 is wrapped by multiple pipe sleeve membrane elements 3 on the outer periphery. For the purpose of fixing, there are two outer ring members 5 disposed on the outer periphery of the two ends of each pipe sleeve membrane element 3, and each pipe sleeve membrane element 3 is individually fastened to the multi-lumen pipeline structure 2 to form the multiple sleeve membrane structure 32. In other embodiments, the integrally formed multi-lumen pipeline structure 2 is wrapped by one pipe sleeve membrane element 3 on the outer periphery. The pipe sleeve membrane element 3 is fixed by three outer ring members 5 to form two sleeve membrane structures 32. In some embodiments, the above-mentioned multiple pipe sleeve membrane element 3 and/or one pipe sleeve membrane element 3 are used to form more than three sleeve membrane structures 32.

In some embodiments, for achieving complete air-tight between the multi-lumen pipeline structure 2 and pipe sleeve membrane element 3, the outer ring members 5 may use adhesive (not shown) for fastening the pipe sleeve membrane element 3 with the multi-lumen pipeline structure 2. This allows the multiple sleeve membrane structures 32 to be inflated smoothly. Or in other embodiments, for achieving complete air-tight between the multi-lumen pipeline structure 2 and the pipe sleeve membrane element 3, the adhesive (not shown) is used directly for fastening the pipe sleeve membrane element 3 to the outer periphery of the multi-lumen pipeline structure 2 without the outer ring members 5, and allowing the multiple sleeve membrane structures 32 to be inflated smoothly.

In some embodiments, the lengths of the fluid flow pipe structures 22 in the multi-lumen pipeline structure 2 are different. This allows different fluid flow pipe structure 22 to connect with different sleeve membrane structures 32 respectively so that the sleeve membrane structures 32 can be inflated and deflated and the degree of inflation and deflation can be adjusted independently.

In some embodiments, the independent connecting structure 24 can be a channel or an opening structure.

In some embodiments, the main joint structure 41 and the sub-joint structure 23 can be formed under heat and pressure by heat inlay or the like. It further makes the pointed end 4 stably jointed with the multi-lumen pipeline structure 2. In other embodiments, the pointed end 4 is a closed structure such as a cone or truncated cone structure, or the like. The closed structure can be solid, hollow or other filling methods. In some embodiments, the pointed end 4 and the multi-lumen pipeline structure 2 are integrally formed by different processes.

The integrally formed multi-lumen pipeline structure 2, the pipe sleeve membrane element 3 and the pointed end 4 are made of soft and bendable materials. The materials can be silicone, latex, plastic (such as PVC, PU, PP, PE, PTFE, etc.) or other biocompatible materials or compositions thereof. This allows the sleeve membrane structures 32 formed by fixing the pipe sleeve membrane element 3 to be inflatable after being filled. The tubular structure 21 of the multi-lumen pipeline structure 2 and the fluid flow pipe structures 22 can be designed to have different lengths and diameters adapted for different body parts to be treated. The sleeve membrane structure 32 may also be designed to have different lengths according to the needs. In an embodiment for esophageal cancer, the catheter apparatus 1 can be designed to have a length of 600-1500 mm, preferably a length of 1200 mm. The outer diameter of the catheter apparatus 1 can be designed to be 1.5-10 mm, preferably 6 mm.

In an embodiment for esophageal cancer, the tubular structure 21 can be designed to have an outer diameter of 2-6 mm, preferably 2.5 mm; the inner diameter can be 1-5 mm, preferably 1.2-2.0 mm, so long as a lumencath for assisting the placement of the radioactive source (not shown) is able to be placed therein.

In an embodiment for esophageal cancer, the lengths of the fluid flow pipe structures 22 and the tubular structure 21 are the same. The inner diameter of the fluid flow pipe structures 22 can be 0.2-3 mm, preferably 0.7 mm. The distance between the centers of the fluid flow pipe structures 22 and the center of the tubular structure 21 can be 0.6-3 mm, preferably 1.8-1.9 mm.

In an embodiment for esophageal cancer, the length of the sleeve membrane structure 32 can be 5-100 mm, preferably 10-40 mm, and 30 mm will be better. It can be chosen to inflate to a diameter of 30 mm or less.

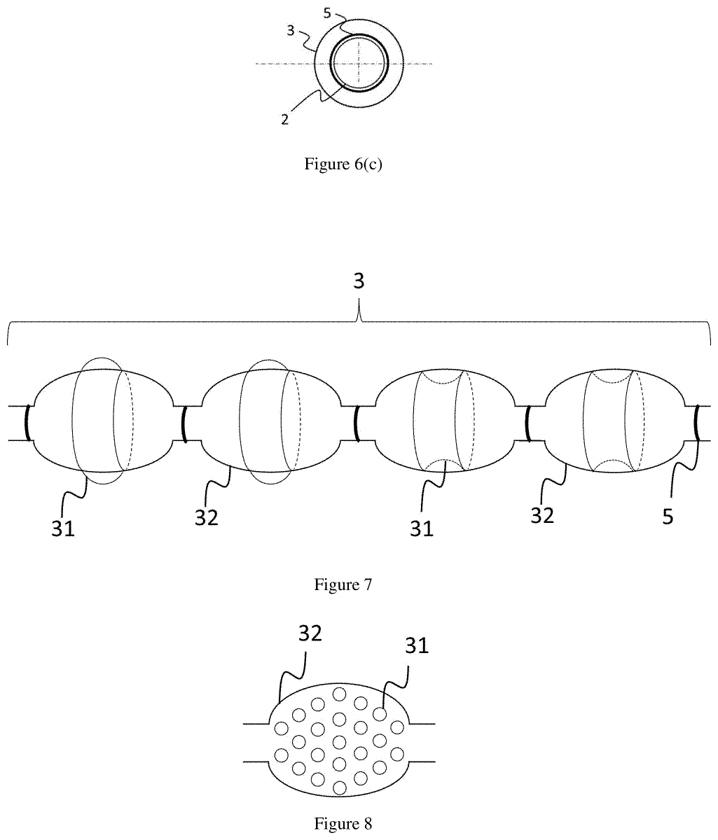

FIGS. 6(a) and 6(b) are illustrative structural diagrams of the sleeve membrane structure 32 according to one embodiment of the present application, respectively. FIG. 6(c) is an illustrative side view of the sleeve membrane structure according to one embodiment of the present application, wherein the two sides of the pipe sleeve membrane element 3 are fastened by two outer ring member 5 to form the sleeve membrane structure 32 surrounding the multi-lumen pipeline structure 2, wherein the sleeve membrane structure 32 can be a cylindrical or a waist drum shaped structure. In some embodiments, the middle segment 33 and the two side segments 34 have their respective membrane thickness. The membrane thickness of the middle segment 33 can be X1, and that of the two side segments 34 can be X2, and X2<X1. In other embodiments, the middle segment 33 has different membrane thicknesses which decreases progressively from the middle to the two sides. That means the middle of the middle segment 33 has a membrane thickness X1 and the two sides of it has a membrane thickness X2. In some embodiments. X2= 1/10 X1.

FIG. 7 to FIG. 12 are illustrative structural diagrams of the strengthening structure 31 according to one embodiment of the present application.

Referring to FIG. 7, in some embodiments, the strengthening structure 31 can be disposed at the inner or outer sides of the pipe sleeve membrane element 3. When the fluid (not shown) is filled and inflated the sleeve membrane structures 32 which are formed after separating the pipe sleeve membrane element 3 by the outer ring members 5, the strengthening structures 31 are used for making each sleeve membrane structure 32 inflate uniformly at a constant speed from an axis to a surrounding radially.

Referring to the illustrative structural diagram of the strengthening structures 31 according to one embodiment of the present application shown in FIG. 7, in some embodiments, the strengthening structures 31 are disposed evenly at the inner or outer side of the pipe sleeve membrane element 3. When the fluid (not shown) is filled and inflated the sleeve membrane structures 32 which are formed after separating the pipe sleeve membrane element 3 by the outer ring members 5, the single strengthening structure 31 located at the center of each sleeve membrane structure 32 uses the axis of the catheter apparatus 1 as a reference for inflating radially at a constant speed. It maintains and ensures that the central radioactive source tube located at the center of the esophagus in order to avoid the production of radiation hotspots.

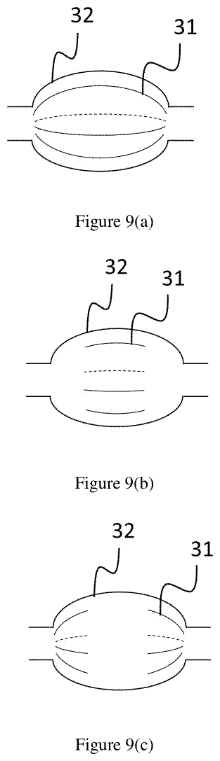

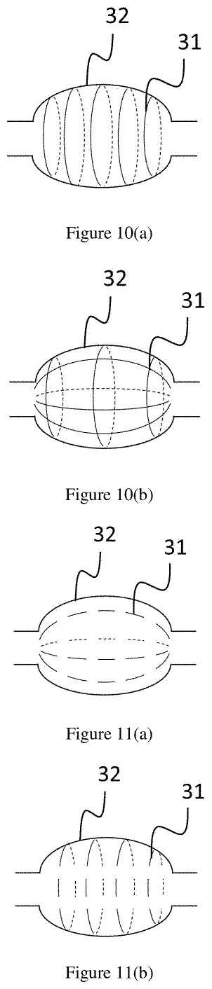

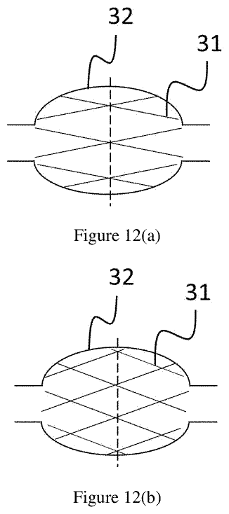

Referring to the illustrative structural diagrams of the strengthening structure 31 according to some embodiments of the present application shown in FIGS. 8-12, wherein the strengthening structures 31 distributed on the sleeve membrane structure 32 can be a dot-shaped, or strip-shaped structure, or other structure. In some embodiments, as shown in FIG. 8, the strengthening structures 31 symmetrically distributed on the sleeve membrane structure 32 are dot-shaped structures. In other embodiments, as shown in FIGS. 9(a) to 9(c), more than two strengthening structures 31 can be strip-shaped structures, and are symmetrically distributed along the long side axis or located at the center or two sides of the sleeve membrane structure 32. In other embodiments, as shown in FIG. 10(a), the strengthening structures 31 can be more than one strip-shaped structure, and parallel and symmetrically distributed along the short side axis of the sleeve membrane structure 32. Or, as shown in FIG. 10(b), the strengthening structures 31 are vertical and criss-crossed to each other and are symmetrically distributed on the entire sleeve membrane structure 32. In other embodiments, as shown in FIG. 11(a) to FIG. 11(b), the strengthening structures 31 are non-continuous and are symmetrically distributed on the sleeve membrane structure 32. In some embodiments, as shown in FIG. 12, the strengthening structures 31 are criss-crossed and are symmetrically distributed on the entire sleeve membrane structure 32. In some embodiments, the symmetric strengthening structures 31 can make the sleeve membrane structure 32 to be inflated evenly.

In some embodiments, the inflated sleeve membrane structure 32 can be in the shape of a ball, cylindrical or other shape (not shown). Since the shape after inflation is not limited, so the basic filling volume of the sleeve membrane structures 32 is also not limited.

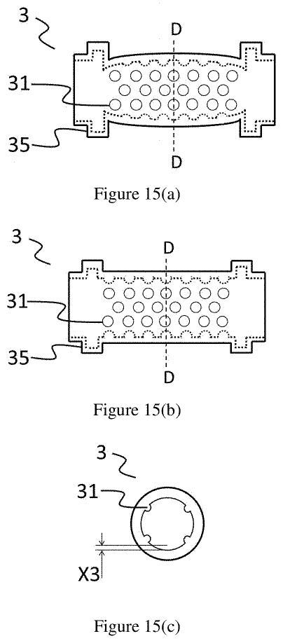

FIG. 13 to FIG. 15 are illustrative side view and cross-sectional view of the strengthening structures 31 and the buffering structures 35 according to some embodiments of the present application.

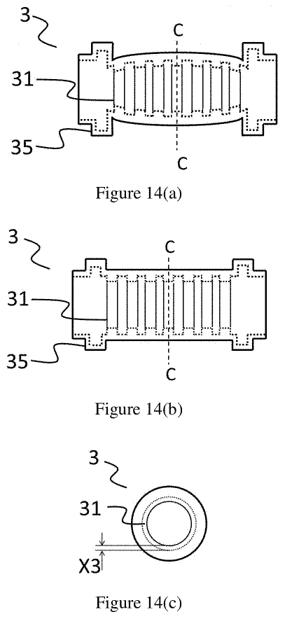

Referring to the strengthening structures 31 shown in FIG. 13 to FIG. 14, in some embodiments, the strip-shaped strengthening structures 31 disposed on the pipe sleeve membrane element 3 can be in geometric shape such as cuboid or cylinder. Referring to the strengthening structures 31 in FIG. 13 to FIG. 15, in other embodiments, the strip-shaped or dot-shaped strengthening structures 31 have a height X3. In some embodiments, X3 is designed to be 0.01-2 mm, preferably 0.1 mm.

Referring to the buffering structure 35 shown in FIG. 13 to FIG. 15, in some embodiments, the buffering structures 35 are disposed at a protruding structure on the outer periphery of the pipe sleeve membrane element 3, and are located at the two sides of each sleeve membrane structure 32. In other embodiments, the buffering structures 35 are disposed at recesses on the outer periphery of the pipe sleeve membrane element 3 (not shown), and are located at the two sides of each sleeve membrane structure 32.

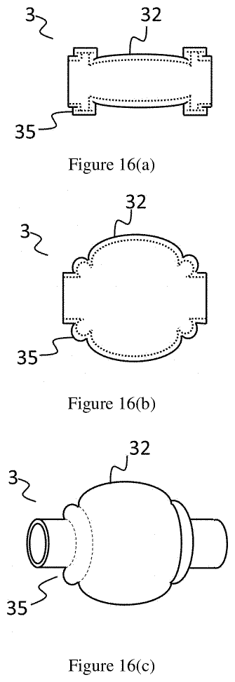

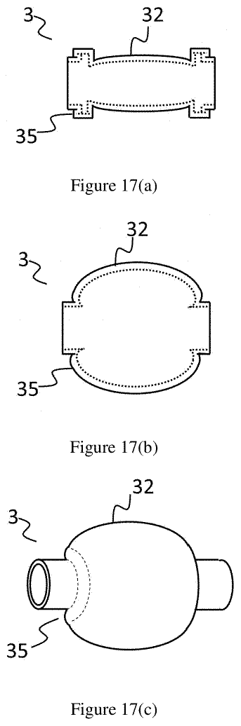

FIG. 16 to FIG. 17 are illustrative structural diagrams of the buffering structures 35 according to some embodiments of the present application.

Referring to the illustrative side view of the buffering structures 35 before inflation according to one embodiment of the present application shown in FIG. 16(a), in some embodiments, the buffering structures 35 are disposed on the outer periphery of the pipe sleeve membrane element 3 and located at the two sides of each sleeve membrane structure 32. Before filling and inflating, both ends of the buffering structures 35 are folded and laid flat on the outer surface of the pipe sleeve membrane element 3, respectively. Referring to the illustrative side view and perspective view of the buffering structures 35 after inflation according to one embodiment of the present application shown in FIGS. 16(b) and 16(c), when filling, tension is released and inflated the sleeve membrane structure 32 before that of the buffering structure 35 using the axis of the catheter apparatus as a reference. Both ends of the buffering structures 35 are then folded and laid flat on the outer surface of the pipe sleeve membrane element 3 and are expanded.

Referring to the illustrative side view of the buffering structures before inflation according to one embodiment of the present application shown in FIG. 17(a), in some embodiments, the buffering structures 35 are disposed on the outer periphery of the pipe sleeve membrane element 3 and located at the two sides of each sleeve membrane structure. One end of the buffering structure 35 is folded and laid flat on the outer side surface of the pipe sleeve membrane element 3 before inflation. Referring to the illustrative side view and perspective view of the buffering structure after inflation according to one embodiment of the present application shown in FIGS. 16(b) and 16(c), when filling, tension is released and inflated the sleeve membrane structure 32 before that of the buffering structure 35 using the axis of the catheter apparatus as a reference. One end of the buffering structures 35 is then folded and laid flat on the outer side surface of the pipe sleeve membrane element 3 and is expanded.

In some embodiments, when the fluid (not shown) is filling in the sleeve membrane structure 32 and making it inflated, tension is released and inflated the sleeve membrane structure 32 before that of the buffering structure 35 by the design of the protruding, recessed, or folding structure of the buffering structure 35. Thus, the entire sleeve membrane structure 32 can maintain evenly distributed tension using the axis of the catheter apparatus as a reference when it is inflating to ensure the uniformity of the sleeve membrane structure 32 during and after inflation.

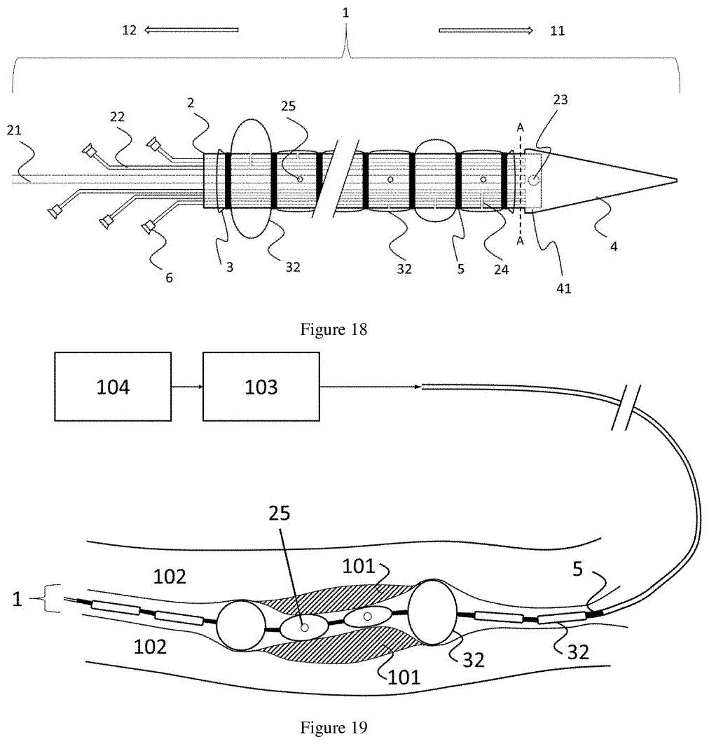

FIG. 18 is an illustrative diagram of the individually inflated sleeve membrane structures 32 of the present application. Each sleeve membrane structure 32 of the present application can be independently controlled in terms of filling with fluid or not and the amount of each filling. Therefore, the degree of inflation and deflation of each sleeve membrane structure 32 can be independently controlled. When the sizes of tumor growth in different segments are different, according to the actual tumor growth situation in the patient's body cavity, the sleeve membrane structures 32 may be inflated by being filled with a smaller amount of fluid at a narrow segment of the body cavity (formed by a relatively larger or more protruding tumor), or the sleeve membrane structure 32 may be inflated to a larger size by being filled with more fluid when the tumor grows more superficially (the esophageal lumen is relatively narrow), thereby achieving the purpose of killing the tumor with less radiation dose, and reducing side effects.

As shown in FIG. 19, after being connected to the afterload-type therapeutic instrument 103, the catheter apparatus 1 (some of the components are omitted) can determine which sleeve membrane structure 32 needs to be inflated and deflated, and the degree of inflation and deflation, according to the size and position of the tumor tissue 101 in the body cavity. Then, the radioactive source 25 is placed and the brachytherapy is carried out. Since the sleeve membrane structures 32 of the catheter apparatus 1 of the present application can be evenly inflated and deflated, the axis of the catheter apparatus 1 can be located at the center of esophagus. When arranging the treatment plan for the patient, it may maintain the radioactive source at the center of the esophagus and avoid the production of radiation hotspots.

The position at which the sleeve membrane structure 32 is inflated and deflated, and the degree of the inflation and deflation are determined according to the image taken by the tumor imaging instrument 104. The tumor imaging instrument 104 includes X-ray imaging, fluoroscope, computed tomography scan, positron tomography scan, single photon emission tomography imaging, nuclear magnetic resonance imaging, and the like.

Below are the installation steps of the technical features of the present invention utilized in the embodiments of esophageal cancer assisting a person having ordinary skill in the art to understand the possible application of the present invention. The invention may be applied in place of other steps of use without departing from the scope of the present application:

The catheter apparatus 1 is placed into the esophagus from the nasal cavity. In the state in which the sleeve membrane structures 32 have not been inflated and deflated, the catheter apparatus 1 can be smoothly placed into the esophagus from the nasal cavity and it is not necessary to be placed from the oral cavity. After the catheter apparatus 1 is placed in the esophagus, it is fixed to the outside of the nostrils by adhesion with a tape.

A lumencath (not shown) is placed in the tubular structure 21 of the catheter apparatus 1 until reaching the end, and the lumencath (not shown) is fixed to the tubular structure 21 by adhesion with a tape.

The open end of the lumencath (not shown) is then connected to the afterload-type therapeutic instrument 103 and a simulated radioactive source that can measure the relative depth of the esophagus and develop a CT image is placed therein.

The reconstructed planar image (scout view image) of that portion of the patient is obtained, the distribution area of the simulated radioactive source is observed, and the tumor areas of the computerized tomographic reconstructed planar image of the treatment planning system are compared to determine the position and degree of inflation of the corresponding sleeve membrane structures 32 of the catheter apparatus 1.

After inflating some of the sleeve membrane structures 32, the computer tomographic image is scanned to confirm that the inflated size is appropriate. If necessary, the size is adjusted and a computerized tomographic image is rescanned after modification.

The computed tomography image is transmitted to the treatment planning system, depicting the location and area of the tumor when the sleeve membrane structures 32 are inflated, as well as depicting the surrounding normal tissue (e.g. lung, heart, spinal cord, etc.).

A 3D treatment plan (dose calculation) is made for the patient's various tumor size and shape to ensure that the tumor area has an adequate dose and that the receiving dose of the normal tissue is within a safe range.

The treatment is performed and the radiation is introduced.

Compared with the prior arts of nasogastric tube brachytherapy, or with the catheters of known technology (such as the Bonvoisin-Gerard Esophageal Applicator product of Elekta, U.S. Patent Publication No. US20170173362A1, U.S. Patent Publication No. US20100185173A1, Chinese Patent Publication No. CN2345224Y, etc.), the multiple and independent sleeve membrane structure 32 can be independently controlled in terms of filling with fluid or not and the amount of each filling due to the designs of the strengthening structures 31 or membrane thickness of the pipe sleeve membrane element 3 of the catheter apparatus 1 of the present application. That makes the sleeve membrane structures 32 to be inflated evenly at a constant speed form the axis to the surrounding radically and keeps the axis of the catheter apparatus 1 in the center of the esophagus. The radioactive source of the tubular structure 21 is placed in the center of the esophagus. This solves the problems of radiation hotspots in the prior art caused by the displacement of radioactive source when the catheter axis is not at the center. Compared with the prior arts, the sleeve membrane structure 32 of the catheter apparatus 1 of present application have enough support in the esophagus by an arbitrary amount of fluid filling and fit the patient's esophagus without limitation of its basic amount of filling. The catheter apparatus 1 of the present application can avoid slippery of the catheter in the esophagus due to the change of patient's position or esophageal peristalsis without extra external fixing stand. Because of the design of the pointed end 4 of the catheter apparatus 1 of the present application, assisting tools (such as endoscopy, guide wire, etc.) are not necessary when the catheter apparatus enters into the esophagus from the nasal cavity through the throat and the patient's discomfort can be reduced. It also avoids the problem of dropping of the pointed end in the patient's cavity during the process of treatment. Besides, compared with prior arts, due to the whole outer diameter of the catheter apparatus 1 of the present application is smaller than 10 mm before inflation, it won't cause the damage or bleeding of the cavity wall by rubbing with the cavity wall when it is in the cavity and increases the smoothness when it is inserted into the patient's narrow cavity. In addition to the catheter apparatus 1 of present application has sufficient sleeve membrane structures 32 (e.g. 8 inflatable and deflatable sleeve membrane structures), even if it is a diffuse tumor, no movement is required after the catheter apparatus is placed, which makes the patient comfortable without anesthesia.

The present invention does not require the aid of the guide wire and the pointed end won't fall off in the cavity and won't change the physician's habit. It can irradiate the entire diffuse tumor in one brachytherapy without placing the catheter and the radioactive source repeatedly, and can avoid changes of the relative position between the catheter and the tumor that is caused by the breathing or movement of the patient. It may save the physician's energy and improve the accuracy of the treatment plan. The present application can maintain evenly distributed tension and ensure the uniformity of the sleeve membrane structures during and after inflation based on the design of the strengthening structures and/or buffering structures. The present application does not need to be placed from the oral cavity for the treatment of esophageal cancer, and it is not necessary to administer anesthesia to the patient. In addition, instead of an external balloon, inflatable and deflatable sleeve membrane structures may not rub against the body cavity wall when it is entering the body cavity which causes discomfort to the patient or even wall damage or bleeding. It ensures that the radioactive source is located in the center of the esophagus and avoids side effect caused by the production of radiation hotspots during brachytherapy. It solves the problems of the existing technology and achieves a better effect.

### SYMBOL DESCRIPTION

Catheter apparatus 1
Distal end direction 11
Proximal end direction 12
Multi-lumen pipeline structure 2
Tubular structure 21
Fluid flow pipe structure 22
Sub-joint structure 23
Independent connecting structure 24
Radioactive source 25
Pipe sleeve membrane element 3
Strengthening structure 31
Sleeve membrane structure 32
Middle segment 33
Two side segments 34
Buffering structure 35
Pointed end 4
Main joint structure 41
Outer ring element 5
Control element 6
Tumor tissue 101
Normal tissue 102
Afterload-type therapeutic instrument 103
Tumor imaging instrument 104
Cross section A-A, B-B, C-C, D-D
Membrane thickness X1, X2, X3
Tumor size GTV
Spread area CTV
Moving deviation area ITV
Treatment boundary area PTV

1. A catheter apparatus, comprising:
an integrally formed multi-lumen pipeline structure, having a proximal end direction and a distal end direction, wherein the multi-lumen pipeline structure comprises a tubular structure and multiple fluid flow pipe structures, the tubular structure and the multiple fluid flow pipe structures are disposed along a first axial direction of the multi-lumen pipeline structure;
at least one pipe sleeve membrane element wrapped around an outer periphery of the multi-lumen pipeline structure, wherein the at least one pipe sleeve membrane element comprises a strengthening structure and/or a buffering structure;
a pointed end, which is jointed to the multi-lumen pipeline structure and is firmly fixed with the multi-lumen pipeline structure; and
a quantity of the pipe sleeve membrane element is more than 1; a quantity of the fluid flow pipe structures is more than 3.

2. The catheter apparatus according to claim 1, wherein the catheter apparatus is further provided with multiple outer ring elements disposed on an outer periphery of the pipe sleeve membrane element, the multiple outer ring elements are used for fastening the pipe sleeve membrane element with the multi-lumen pipeline structure to form multiple sleeve membrane structures, wherein each sleeve membrane structure is a cylindrical or waist drum shaped structure surrounding the multi-lumen pipeline structure, a quantity of the sleeve membrane structures is more than 3.

3. (canceled)

4. The catheter apparatus according to claim 2, wherein each sleeve membrane structure has a membrane thickness, a middle segment and two side segments, the membrane thickness decreases progressively from the middle segment to the two side segments.

5. (canceled)

6. The catheter apparatus according to claim 1, wherein the pointed end is integrally formed with the multi-lumen pipeline structure, wherein the pointed end is a cone or truncated cone structure jointed with the multi-lumen pipeline structure.

7. (canceled)

8. The catheter apparatus according to claim 1, wherein the pointed end is a closed structure, and the pointed end further comprises a material which can absorb radiation.

9. The catheter apparatus according to claim 1, wherein the pointed end comprises a main joint structure which is used for fixing to a sub-joint structure of the multi-lumen pipeline structure, wherein the main joint structure and the sub-joint structure are corresponding to each other and are in the form of a latch, a buckle or a screw.

10. (canceled)

11. The catheter apparatus according to claim 2, wherein the strengthening structure is disposed on an inner or outer side of the pipe sleeve membrane element, making each of the sleeve membrane structures to be inflated uniformly at a constant speed from an axis to a surrounding radially.

12. The catheter apparatus according to claim 1, wherein the strengthening structure is at least one strip-shaped structure or multiple dot-shaped structures distributed on the sleeve membrane structures.

13. The catheter apparatus according to claim 12, wherein the strip-shaped structure is a symmetrical, parallel, criss-cross and/or non-continuous structure.

14. The catheter apparatus according to claim 2, wherein the buffering structure is a recessed, protruding or folding structure disposed on the outer periphery of the pipe sleeve membrane element, making the sleeve membrane structures release pressure uniformly during beginning of inflation.

15. The catheter apparatus according to claim 1, wherein the multiple fluid flow pipe structures are provided with a control element in the proximal end direction, and the control element is configured for independently inflating and deflating each sleeve membrane structure connected to the fluid flow pipe structure in the distal end direction.

16. The catheter apparatus according to claim 2, wherein the multiple fluid flow pipe structures are provided with multiple control elements in the proximal end direction, and the multiple control elements are disposed individually and independently at the multiple fluid flow pipe structures in the proximal end direction; the control element is configured for independently inflating and deflating each sleeve membrane structure connected to the fluid flow pipe structure in a distal end position.

17. The catheter apparatus according to claim 2, wherein the multiple fluid flow pipe structures each are provided with an independent connecting structure which are connected with different positions of the sleeve membrane structures respectively, transferring fluid from the fluid flow pipe structures to different positions of the sleeve membrane structures through respective independent connecting structures, wherein the independent connecting structure is a channel or an opening structure.

18. (canceled)

19. (canceled)

20. (canceled)

21. (canceled)

22. (canceled)

23. A brachytherapy system, comprising:
an afterload-type therapeutic instrument;
a catheter apparatus according to claim 1 connected to the afterload-type therapeutic instrument;
a radiation therapeutic source, released from the afterload-type therapeutic instrument to the tubular structure of the catheter apparatus;
a tumor imaging instrument, wherein the afterload-type therapeutic instrument releases the radiation therapeutic source to the tubular structure according to determination by the tumor imaging instrument, wherein the tumor imaging instrument comprises one or more of X-ray imaging, fluoroscope, computed tomography scan, positron tomography scan, single photon emission tomography imaging, and nuclear magnetic resonance imaging; and
the brachytherapy system is used for treatment of esophageal cancer or other intracavitary tumors.

24. A brachytherapy system, comprising:
an afterload-type therapeutic instrument;
a catheter apparatus according to claim 2 connected to the afterload-type therapeutic instrument;
a radiation therapeutic source, released from the afterload-type therapeutic instrument to the tubular structure of the catheter apparatus;
a tumor imaging instrument, wherein the afterload-type therapeutic instrument releases the radiation therapeutic source to the tubular structure according to determination by the tumor imaging instrument, wherein the tumor imaging instrument comprises one or more of X-ray imaging, fluoroscope, computed tomography scan, positron tomography scan, single photon emission tomography imaging, and nuclear magnetic resonance imaging; and
the brachytherapy system is used for treatment of esophageal cancer or other intracavitary tumors.

25. A brachytherapy system, comprising:
an afterload-type therapeutic instrument;
a catheter apparatus according to claim 4 connected to the afterload-type therapeutic instrument;
a radiation therapeutic source, released from the afterload-type therapeutic instrument to the tubular structure of the catheter apparatus;
a tumor imaging instrument, wherein the afterload-type therapeutic instrument releases the radiation therapeutic source to the tubular structure according to determination by the tumor imaging instrument, wherein the tumor imaging instrument comprises one or more of X-ray imaging, fluoroscope, computed tomography scan, positron tomography scan, single photon emission tomography imaging, and nuclear magnetic resonance imaging; and
the brachytherapy system is used for treatment of esophageal cancer or other intracavitary tumors.

26. A brachytherapy system, comprising:
an afterload-type therapeutic instrument;
a catheter apparatus according to claim 6 connected to the afterload-type therapeutic instrument;
a radiation therapeutic source, released from the afterload-type therapeutic instrument to the tubular structure of the catheter apparatus;
a tumor imaging instrument, wherein the afterload-type therapeutic instrument releases the radiation therapeutic source to the tubular structure according to determination by the tumor imaging instrument, wherein the tumor imaging instrument comprises one or more of X-ray imaging, fluoroscope, computed tomography scan, positron tomography scan, single photon emission tomography imaging, and nuclear magnetic resonance imaging; and
the brachytherapy system is used for treatment of esophageal cancer or other intracavitary tumors.

27. A brachytherapy system, comprising:
an afterload-type therapeutic instrument;
a catheter apparatus according to claim 11 connected to the afterload-type therapeutic instrument;
a radiation therapeutic source, released from the afterload-type therapeutic instrument to the tubular structure of the catheter apparatus;
a tumor imaging instrument, wherein the afterload-type therapeutic instrument releases the radiation therapeutic source to the tubular structure according to determination by the tumor imaging instrument, wherein the tumor imaging instrument comprises one or more of X-ray imaging, fluoroscope, computed tomography scan, positron tomography scan, single photon emission tomography imaging, and nuclear magnetic resonance imaging; and
the brachytherapy system is used for treatment of esophageal cancer or other intracavitary tumors.

28. A brachytherapy system, comprising:
an afterload-type therapeutic instrument;
a catheter apparatus according to claim 12 connected to the afterload-type therapeutic instrument;
a radiation therapeutic source, released from the afterload-type therapeutic instrument to the tubular structure of the catheter apparatus;
a tumor imaging instrument, wherein the afterload-type therapeutic instrument releases the radiation therapeutic source to the tubular structure according to determination by the tumor imaging instrument, wherein the tumor imaging instrument comprises one or more of X-ray imaging, fluoroscope, computed tomography scan, positron tomography scan, single photon emission tomography imaging, and nuclear magnetic resonance imaging; and
the brachytherapy system is used for treatment of esophageal cancer or other intracavitary tumors.

29. A brachytherapy system, comprising:
an afterload-type therapeutic instrument;
a catheter apparatus according to claim 14 connected to the afterload-type therapeutic instrument;
a radiation therapeutic source, released from the afterload-type therapeutic instrument to the tubular structure of the catheter apparatus;
a tumor imaging instrument, wherein the afterload-type therapeutic instrument releases the radiation therapeutic source to the tubular structure according to determination by the tumor imaging instrument, wherein the tumor imaging instrument comprises one or more of X-ray imaging, fluoroscope, computed tomography scan, positron tomography scan, single photon emission tomography imaging, and nuclear magnetic resonance imaging; and
the brachytherapy system is used for treatment of esophageal cancer or other intracavitary tumors.
